(19) United States
(12) Patent Application Publication
(10) Pub. No.: US 2007/0105923 A1
(43) Pub. Date: May 10, 2007

(54) Substantially pure olmesartan medoxomil and processes for its preparation

(75) Inventors: Bobba Venkata Siva Kumar, Koper Khairne (IN); Sanjay Anantha Kale, Airoli (IN); Raju Baban Choudhari, Kalyan (west) (IN); Nitin Sharad Chandra Pradhan, Thane (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/521,166

(22) Filed: Sep. 14, 2006

Related U.S. Application Data
(60) Provisional application No. 60/812,490, filed on Jun. 9, 2006.
(60) Provisional application No. 60/724,412, filed on Oct. 7, 2005.

(30) Foreign Application Priority Data
Feb. 16, 2006 (IN) 222/MUM/2006
Sep. 14, 2005 (IN) 1109/MUM/2005

(51) Int. Cl. A61K 31/4178; C07D 403/14

(52) U.S. Cl. 514/381; 548/253

(57) ABSTRACT

A process for purifying olmesartan medoxomil is provided comprising (a) dissolving olmesartan medoxomil in a solvent system comprising a ketone and at least one solvent selected from the group consisting of an alcohol-containing solvent, an ester-containing solvent and mixtures thereof to obtain a solution; and (b) recovering substantially pure olmesartan medoxomil. Also disclosed is substantially pure olmesartan medoxomil and pharmaceutical compositions containing same.

### PRIORITY

This application claims the benefit under 35 U.S.C. §119 to U.S. Provisional Application No. 60/812,490, filed on Jun. 9, 2006, and entitled “SUBSTANTIALLY PURE OLMESARTAN MEDOXOMIL AND PROCESS FOR ITS PREPARATION” and to Indian Provisional Application 222/MUM/2006, filed on Feb. 16, 2006, and entitled “SUBSTANTIALLY PURE OLMESARTAN MEDOXOMIL AND PROCESS FOR ITS PREPARATION”, and to U.S. Provisional Application No. 60/724,412, filed on Oct. 7, 2005, and entitled “SUBSTANTIALLY PURE OLMESARTAN MEDOXOMIL AND PROCESS FOR ITS PREPARATION”, and to Indian Provisional Application 1109/MUM/2005, filed on Sep. 14, 2005, and entitled “SUBSTANTIALLY PURE OLMESARTAN MEDOXOMIL AND PROCESS FOR ITS PREPARATION”, the contents of each of which are incorporated by reference herein.

### BACKGROUND OF THE INVENTION

1. Technical Field

The present invention generally relates to substantially pure olmesartan medoxomil and processes for its preparation.

2. Description of the Related Art

Olmesartan medoxomil, also known as 4-(1-hydroxy-1-methylethyl)-2-propyl-1-[[2′-(1H-tetrazol-5-yl)[1,1′-biphenyl]-4-yl]methyl]-1H-imidazole-5-carboxylic acid (5-methyl-2-oxo-1,3-dioxol-4-yl)methyl ester, is represented by the structure of Formula I. 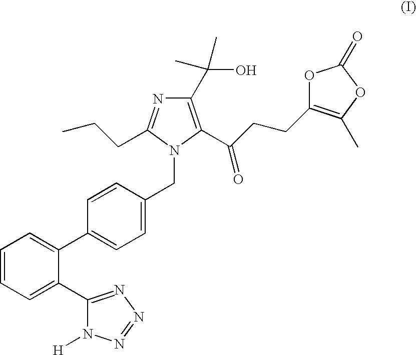
Olmesartan medoxomil is a prodrug that is hydrolyzed to olmesartan during absorption from the gastrointestinal tract. Olmesartan is a selective AT₁ subtype angiotensin II receptor antagonist. Angiotensin II is formed from angiotensin I in a reaction catalyzed by angiotensin converting enzyme (ACE, kinase II). Angiotensin II is the principal pressor agent of the renin-angiotensin system, with effects that include vasoconstriction, stimulation of synthesis and release of aldosterone, cardiac stimulation and renal reabsorption of sodium. Olmesartan blocks the vasoconstrictor effects of angiotensin II by selectively blocking the binding of angiotensin II to the AT1 receptor in vascular smooth muscle. Olmesartan medoxomil is indicated for hypertension and is commercially sold under the trade name Benicar®. See, e.g., The Merck Index, Thirteenth Edition, 2001, pp. 1223-24, monograph 6909; and Physician's Desk Reference, “Benicar,” 60^{th} Edition, pp. 2850-2852 (2005).

U.S. Pat. Nos. 5,616,599 and 5,646,171 disclose 1-biphenylmethylimidazole compounds such as olmesartan medoxomil and processes for their preparation.

U.S. Pat. No. 6,040,454 discloses a process for preparing 1-(tetrazolylbiphenylmethyl)imidazole derivatives such as olmesartan medoxomil by reacting a nitrile with an inorganic azide salt in an aromatic hydrocarbon solvent and in the presence of an amine salt.

Japanese Unexamined Patent Publication No. 53,489/1995 discloses a process for preparing 1-(tetrazolylbiphenylmethyl)imidazole derivative by reacting a 1-(cyanobiphenylmethyl)imidazole derivative with an organotin azide in an aromatic hydrocarbon solvent or a polar solvent and isolating the product olmesartan medoxomil.

A problem associated with the use of inorganic and organic azide compounds in processes for preparing 1-(tetrazolylbiphenylmethyl)-imidazole derivatives such as olmesartan medoxomil is that in order to achieve the high efficiency of the reaction on an industrial scale, it is necessary to find a purification method to increase the purity of the resulting product in order to limit, for example, the degradation of olmesartan medoxomil to olmesartan.

WO 2006/029057 discloses a process for purifying olmesartan medoxomil which includes the steps of (a) providing a solution of olmesartan medoxomil in a C₃-C₆ ketone, preferably acetone; (b) adding water to the solution to precipitate the purified olmesartan medoxomil; and (c) recovering purified olmesartan medoxomil.

Accordingly, it would be desirable to provide an improved process to prepare olmesartan medoxomil in relatively high purity that eliminates and/or reduces the problems of known processes on a commercial scale and in a convenient and cost efficient manner.

### SUMMARY OF THE INVENTION

In accordance with one embodiment of the present invention, a process for purifying olmesartan medoxomil is provided comprising (a) dissolving olmesartan medoxomil in a solvent system comprising a ketone and at least one solvent selected from the group consisting of an alcohol-containing solvent, an ester-containing solvent and mixtures thereof to obtain a solution; and (b) recovering substantially pure olmesartan medoxomil.

In accordance with a second embodiment of the present invention, a process for purifying olmesartan medoxomil is provided comprising (a) providing a solution comprising olmesartan medoxomil in a solvent system comprising a ketone and at least one other solvent selected from the group consisting of an alcohol-containing solvent, an ester-containing solvent and mixtures thereof; (b) heating the solution to an elevated temperature; (c) cooling the solution to induce precipitation of the olmesartan medoxomil; and (d) recovering substantially pure olmesartan medoxomil.

In accordance with a third embodiment of the present invention, a process for purifying olmesartan medoxomil is provided comprising (a) dissolving olmesartan medoxomil in a solvent system comprising a halogenated hydrocarbon-containing solvent and an ester-containing solvent to obtain a solution; and (b) recovering substantially pure olmesartan medoxomil.

In accordance with a fourth embodiment of the present invention, olmesartan medoxomil having a purity greater than or equal to about 98% is provided.

In accordance with a fifth embodiment of the present invention, olmesartan medoxomil having a purity greater than or equal to about 98% and less than about 2% of free olmesartan is provided.

In accordance with a sixth embodiment of the present invention, substantially pure olmesartan medoxomil is provided having a purity of greater than or equal to about 99.5% and less than about 2% of free olmesartan.

In accordance with a seventh embodiment of the present invention, a pharmaceutical composition is provided comprising a therapeutically effective amount of olmesartan medoxomil having a purity greater than or equal to about 98%

Definitions

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

The term “diluent” or “filler” as used herein is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starched thereof, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, polyvinylpyrrolidone (PVP), tyloxapol (a nonionic liquid polymer of the alkyl aryl polyether alcohol type, also known as superinone or triton), combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### DETAILED DESCRIPTION OF THE PREFERRED EMBODIMENTS

One embodiment of the present invention is directed to substantially pure olmesartan medoxomil. Another embodiment of the present invention provides processes for the formation of substantially pure olmesartan medoxomil and pharmaceutically acceptable salts thereof from crude olmesartan medoxomil. In one embodiment, a process for purifying olmesartan medoxomil of the present invention includes at least (a) dissolving olmesartan medoxomil in a solvent system containing at least a ketone and at least one solvent selected from the group consisting of an alcohol-containing solvent, an ester-containing solvent and mixtures thereof to obtain a solution; and (b) recovering substantially pure olmesartan medoxomil.

In step (a) of the process of the present invention, olmesartan medoxomil is added to a solvent system comprising a ketone and at least one solvent selected from the group consisting of an alcohol-containing solvent, an ester-containing solvent and mixtures thereof to form a solution. Generally, olmesartan medoxomil is known and may be prepared according to known techniques. See, e.g., U.S. Pat. Nos. 5,616,599; 5,744,612 and 6,040,454, the contents of which are incorporated herein by reference. The solvent selected from the group consisting of an alcohol-containing solvent and/or an ester-containing solvent advantageously increase the solubility of the olmesartan medoxomil in the solution.

Suitable ketones include ketones having from 3 to about 12 carbon atoms and preferably 3 to about 6 carbon atoms. Representative examples include, but are not limited to, acetone, methyl ethyl ketone, diethyl ketone, methyl propyl ketone, methyl isopropyl ketone, ethyl propyl ketone, ethyl isopropyl ketone, dipropyl ketone, diisopropyl ketone, methyl butyl ketone, methyl isobutyl ketone, methyl sec butyl ketone, methyl tert-butyl ketone, ethyl butyl ketone, ethyl isobutyl ketone, ethyl sec-butyl ketone, ethyl tert-butyl ketone, propyl butyl ketone, isopropyl butyl ketone, propyl isobutyl ketone, propyl sec-butyl ketone, propyl tert butyl ketone, isopropyl isobutyl ketone, isopropyl sec-butyl ketone, isopropyl tert-butyl ketone, dibutyl ketone, diisobutyl ketone, di-sec-butyl ketone, di-tert-butyl ketone, butyl isobutyl ketone, butyl sec-butyl ketone, butyl tert-butyl ketone, isobutyl sec-butyl ketone, isobutyl tert-butyl ketone, sec-butyl tert-butyl ketone, 5-heptanone, 5-methyl-2-hexanone(methyl isoamyl ketone), 4-methyl-2-hexanone, 3-methyl-2-hexanone, 3,4-dimethyl-2-pentanone, 3,3-dimethyl-2-pentanone, 4,4-dimethyl-2-pentanone, 3-octanone, 4-methyl-3-heptanone, 5-methyl-3-heptanone, 6-methyl-3-heptanone, 4,4-dimethyl-3-hexanone, 4,5-dimethyl-3-hexanone, 5,5-dimethyl-3-hexanone, 4-nonanone, 5-methyl-4-octanone, 6-methyl-4-octanone, 7-methyl-4-octanone, 5,5-dimethyl-4-neptanone, 5,6-dimethyl-4-heptanone, 6,6-dimethyl-4-heptanone, 2-undecanone, cyclopropanone, cyclobutanone, cyclopentanone, cyclohexanone, cycloheptanone, cyclooctanone, cyclononanone, cyclodecanone, cycloundecanone, cyclododecanone and the like and mixtures thereof. Preferably, the ketone is acetone.

Suitable alcohol-containing solvents include, but are not limited to, primary, secondary and tertiary alcohols having 1 to about 12 carbon atoms such as aliphatic and aromatic alcohols. Representative examples of such alcohols include methanol, ethanol, isopropanol, butanol and the like and mixtures thereof. Suitable ester-containing solvents include, but are not limited to, ethyl acetate, isopropyl acetate, methyl acetoacetate, ethyl acetoacetate and the like and mixtures thereof.

The solution provided in step (a) will ordinarily contain one or more ketones at a concentration ranging from about 5 to about 10% weight per volume (w/v) and preferably from about 5 to about 6% w/v in the solution, and a second solvent, i.e., an alcohol-containing solvent, an ester-containing solvent and mixtures thereof, at a concentration ranging from about 10 to about 20% w/v and preferably from about 11 to about 12% w/v in the solution.

The solution containing olmesartan medoxomil and the solvent system may be heated to an elevated temperature to obtain a homogeneous solution of the olmesartan medoxomil and solvent mixture. As used herein, the term “elevated temperature” means a temperature above about 30° C. In one embodiment, the mixture can be heated to a temperature ranging from about 30° C. to about 100° C. In another embodiment, the mixture can be heated to a temperature ranging about 50° C. to 75° C. and preferably from about 55° C. to about 70° C. The solution can be heated for a time period sufficient to obtain a clear solution. The time period will ordinarily range from about 15 minutes to about 10 hours and preferably from about 1 hour to about 2 hours.

After the olmesartan medoxomil has been dissolved in the solvent system, precipitation of a solid enriched in olmesartan medoxomil from the solution can be induced, e.g., by cooling. In this manner, cooling will result in precipitation of a solid enriched in olmesartan medoxomil relative to the olmesartan medoxomil dissolved in the solvent system. Generally, cooling includes passive cooling by cessation of active heating. In one embodiment, the solution can be cooled to a reduced temperature below that of the heated solution. As used herein, the term “reduced temperature” means a temperature below about 30° C. In one embodiment, the solution can be cooled to a temperature ordinarily ranging from about 0 to about 30° C. In another embodiment, the solution can be cooled to a temperature ordinarily ranging from about 10° C. to about 25° C. As one skilled in the art will readily appreciate, precipitation can also be induced with the aid of an anti-solvent, optionally with cooling. Suitable anti-solvents include, but are not limited to, ether-containing solvents, e.g., tetrahydrofuran and the like; aromatic hydrocarbon solvents, e.g., toluene, xylene and the like; aliphatic hydrocarbon solvents, e.g., n-hexane heptane and the like; cycloaliphatic-hydrocarbon solvents, e.g., cyclohexane and the like, as well as any other known anti-solvents and mixtures thereof. Alternatively, the solution may be seeded with seed crystals of the pure product to advantageously enhance the crystallization of the pure product.

Following precipitation of the solid enriched in olmesartan medoxomil from the solution, the solid is separated from the solution depleted of olmesartan medoxomil to obtain purified olmesartan medoxomil. The precipitated olmesartan medoxomil can be separated by any conventional technique for removing a solid from a liquid, e.g., distillation, distillation under vacuum, filtration, filtration under vacuum, decantation, and/or centrifugation. In addition, the solid obtained from the separation step can optionally be washed and dried, such as is illustrated in the examples.

The product thus obtained may be further or additionally dried to achieve the desired residual solvent values. For example, the product may be further or additionally dried in a tray drier, or dried under vacuum and/or in a fluid bed drier. If desired, the solution containing olmesartan medoxomil can be treated with activated charcoal and filtered while hot or the slurry containing the pure olmesartan medoxomil may be cooled prior to filtration.

Another embodiment of the present invention provides a process for purifying olmesartan medoxomil which includes at least (a) dissolving olmesartan medoxomil in a solvent system containing at least a halogenated hydrocarbon-containing solvent and an ester-containing solvent to obtain a solution; and (b) recovering substantially pure olmesartan medoxomil. Process conditions can be those as discussed hereinabove.

Suitable halogenated hydrocarbon-containing solvents include, but are not limited to, dichloromethane, chloroform, ethylene dichloride, and the like and mixtures thereof. Suitable ester-containing solvents can be those discussed herein above. In general, the solution provided in step (a) will ordinarily contain one or more halogenated hydrocarbon-containing solvents at a concentration ranging from about 2 to about 4% w/v and preferably from about 2.5 to about 3.5% w/v in the solution, and the ester-containing solvent at a concentration ranging from about 8 to about 12% w/v and preferably from about 9 to about 10% w/v in the solution.

By performing the purification processes of the present invention, substantially pure olmesartan medoxomil can be prepared with a degree of purity as determined by HPLC of greater than or equal to about 98%, preferably greater than or equal to about 99% and most preferably greater than or equal to about 99.5% as compared to the crude product. Also, the content of free olmesartan in the final product as determined by HPLC can be at a level of less than about 2%, preferably less than about 1%, more preferably less than about 0.5% and most preferably less than about 0.1% as compared to the crude product. Moreover, the substantially pure olmesartan medoxomil may be obtained substantially free of any unknown impurity, e.g., a content of less than about 0.1% of impurities. The crude olmesartan medoxomil for use herein can have a purity as determined by HPLC which can ordinarily vary in the range of from about 92% to less than 98%.

Another aspect of the present invention is directed to a pharmaceutical composition containing at least the substantially pure olmesartan medoxomil disclosed herein and at least one pharmaceutically acceptable excipient. Such pharmaceutical compositions may be administered to a mammalian patient in any dosage form, e.g., liquid, powder, elixir, injectabe solution, etc.

In one embodiment, the substantially pure olmesartan medoxomil or pharmaceutically acceptable salt thereof disclosed herein for use in the pharmaceutical compositions of the present invention can have a D₅₀ and D₉₀ particle size of less than about 400 microns, preferably less than about 200 microns, more preferably less than about 150 microns, still more preferably less than about 50 microns and most preferably less than about 15 microns. It is noted the notation Dₓ means that X% of the particles have a diameter less than a specified diameter D. Thus, a D₅₀ of about 400 microns means that 50% of the micronized substantially pure olmesartan medoxomil particles in a composition have a diameter less than about 400 microns. The term “micronization” used herein means any process or methods by which the size of the particles is reduced. For example, the particle sizes of the olmesartan medoxomil or pharmaceutically acceptable salt thereof disclosed herein can be obtained by any milling, grinding, micronizing or other particle size reduction method known in the art to bring the solid state form of the olmesartan medoxomil or pharmaceutically acceptable salt thereof into any of the foregoing desired particle size range. As also used herein, olmesartan medoxomil particles with reduced size are referred to as “micronized particles of olmesartan medoxomil or pharmaceutically acceptable salt thereof” or “micronized olmesartan medoxomil or pharmaceutically acceptable salt thereof”.

The dosage forms may contain the substantially pure olmesartan medoxomil disclosed herein or, alternatively, may contain the substantially pure olmesartan medoxomil as part of a composition. Whether administered in pure form or in a composition, the substantially pure olmesartan medoxomil may be in any form, for example, compacted tablets, powder suspensions, capsules, and the like. The compositions of the present invention can be administered to humans and animals in such dosage forms as oral, rectal, parenteral (intravenous, intramuscular, or subcutaneous), intracistemal, intravaginal, intraperitoneal, local (powders, ointments or drops), ophthalmic, transdermal, or sublingual forms or as a buccal or nasal spray. Oral dosage forms include, but are not limited to, pills, troches, sachets, suspensions, powders, lozenges, elixirs, tablets, capsules (including soft gel capsules), ovules, solutions, and the like which may contain flavoring or coloring agents, for immediate-, delayed-, modified-, or controlled-release such as sustained-, dual-, or pulsatile delivery applications. The substantially pure form of olmesartan medoxomil disclosed herein also may be administered as suppositories, ophthalmic ointments and suspensions, and parenteral suspensions, which are administered by other routes. The most preferred route of administration of the olmesartan medoxomil of the present invention is oral. The pharmaceutical compositions may further contain one or more pharmaceutically acceptable excipients as described herein.

The active ingredient of the invention may also be administered via fast dispersing or fast dissolving dosage forms or in the form of a high energy dispersion or as coated particles. Suitable pharmaceutical composition of the invention may be in coated or uncoated form as desired.

Capsule dosages will contain the solid composition within a capsule which may be coated with gelatin. Tablets and powders may also be coated with an enteric coating. The enteric-coated powder forms may have coatings comprising phthalic acid cellulose acetate, hydroxypropylmethyl cellulose phthalate, polyvinyl alcohol phthalate, carboxymethylethylcellulose, a copolymer of styrene and maleic acid, a copolymer of methacrylic acid and methyl methacrylate, and -like materials, and if desired, they may be employed with suitable plasticizers and/or extending agents. A coated tablet may have a coating on the surface of the tablet or may be a tablet comprising a powder or granules with an enteric coating.

Tabletting compositions may have few or many components depending upon the tabletting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

Actual dosage levels of the substantially pure form of olmesartan medoxomil of the present invention in the pharmaceutical compositions may be varied to obtain an amount of the substantially pure form of olmesartan medoxomil disclosed herein that is effective to obtain a desired therapeutic response for a particular composition and method of administration. The selected dosage level therefore depends upon such factors as, for example, the desired therapeutic effect, the route of administration, the desired duration of treatment, and other factors. The total daily dose of the substantially pure form of olmesartan medoxomil of this invention administered to a host in single or divided dose and can vary widely depending upon a variety of factors including, for example, the body weight, general health, sex, diet, time and route of administration, rates of absorption and excretion, combination with other drugs, the severity of the particular condition being treated, etc.

The following examples are provided to enable one skilled in the art to practice the invention and are merely illustrative of the invention. The examples should not be read as limiting the scope of the invention as provided in the features and advantages.

Experimental

The purity was measured by high performance liquid chromatography (HPLC) under the following conditions:
Column: C18, Phenomenex Luna, 250×4.6 mm, 5 μ
Moving phase: A-Buffer—3.4 g potassium dihydrogen phosphate in 1000 ml water B-Acetonitrile
Gradient: Buffer+Acetonitrile
Detector: UV, 225 nm
Flow rate: 1 ml/min
Retention time: 13 minutes

### EXAMPLE 1

Preparation of Pure Olmesartan Medoxomil

Into a four-neck 500 ml flask equipped with a mechanical stirring condenser and thermometer and charged with acetone (560 ml) and ethyl acetate (1150 ml) was added crude olmesartan medoxomil (100 g). The suspension was slowly heated to a temperature ranging from about 55° C. to about 65° C. and maintained for about 1 to 2 hours to obtain a clear solution. The solution was then stirred at the same temperature for 1 hour and any insolubles were removed by filtration. The clear filtrate was slowly cooled to room temperature and then further cooled to 10° C. to 20° C. The precipitate was filtered on a Buchner funnel and washed with ethyl acetate (50 ml) and was a solid enriched in olmesartan medoxomil. The filtered product was dried to provide pure olmesartan medoxomil (65 g) having a purity of greater than 99.7% and a content of free olmesartan of 0.08% as determined by HPLC

### EXAMPLE 2

Preparation of Pure Olmesartan Medoxomil

Into a four-neck 500 ml flask equipped with a mechanical stirring condenser and thermometer and charged with isopropyl alcohol (1200 ml) and acetone (800 ml) was added crude olmesartan medoxomil (100 g). The suspension was slowly heated to a temperature ranging from about 55° C. to about 65° C. and maintained for about 1 to 2 hours to obtain a clear solution. The solution was then stirred at the same temperature for 1 hour and any insolubles were removed by filtration. The clear filtrate was slowly cooled to room temperature and then further cooled to 10° C. to 20° C. The precipitate was filtered on a Buchner funnel and washed with ethyl acetate (50 ml) and was a solid enriched in olmesartan medoxomil. The filtered product was dried to provide pure olmesartan medoxomil (70 g) having a purity of 99.7% and no free olmesartan was detected as determined by HPLC.

### EXAMPLE 3

Preparation of Pure Olmesartan Medoxomil

Into a four-neck 500 ml flask equipped with a mechanical stirring condenser and thermometer and charged with ethyl acetate (500 ml) and dichloromethane (1500 ml) was added crude olmesartan medoxomil (50 g). The suspension was slowly heated to a temperature ranging from about 50° C. to about 55° C. and maintained for about 1 to 2 hours to obtain a clear solution. The solution was then stirred at the same temperature for 1 hour and any insolubles were removed by filtration. The clear filtrate was slowly cooled to room temperature and then further cooled to 10° C. to 20° C. The precipitate was filtered on a Buchner funnel and washed with ethyl acetate (50 ml) and was a solid enriched in olmesartan medoxomil. The filtered product was dried to provide pure olmesartan medoxomil (36 g) having a purity of 99.6% and no free olmesartan was detected as determined by HPLC.

### EXAMPLE 4

Preparation of Pure Olmesartan Medoxomil

Into a four-neck 500 ml flask equipped with a mechanical stirring condenser and thermometer and charged with acetone (560 ml) and ethyl acetate (1150 ml) was added crude olmesartan medoxomil (100 g). The suspension was slowly heated to a temperature ranging from about 55° C. to about 65° C. and maintained for about 1 to 2 hours to obtain a clear solution. The solution was then stirred at the same temperature for 1 hour and any insolubles were removed by filtration. The clear filtrate was slowly cooled to room temperature and then further cooled to 10C to 20° C. The precipitate was filtered on a Buchner funnel and washed with ethyl acetate (50 ml) and was a solid enriched in olmesartan medoxomil. The filtered product was dried to provide pure olmesartan medoxomil (65 g) having a purity of 99.6%, and a content of free olmesartan of 0.2% as determined by HPLC.

### EXAMPLE 5

Preparation of Pure Olmesartan Medoxomil

Into a four-neck 500 ml flask equipped with a mechanical stirring condenser and thermometer and charged with isopropyl alcohol (1200 ml) and acetone (800 ml) was added crude olmesartan medoxomil (100 g). The suspension was slowly heated to a temperature ranging from about 55° C. to about 65° C. and maintained for about 1 to 2 hours to obtain a clear solution. The solution was then stirred at the same temperature for 1 hour and any insolubles were removed by filtration. The clear filtrate was slowly cooled to room temperature and then further cooled to 10° C. to 20° C. The precipitate was filtered on a Buchner funnel and washed with ethyl acetate (50 ml) and was a solid enriched in olmesartan medoxomil. The filtered product was dried to provide pure olmesartan medoxomil (70 g) having a purity of 99.65% and a content of free olmesartan of 0.11% as determined by HPLC.

### EXAMPLE 6

Preparation of Pure Olmesartan Medoxomil

Into a four-neck 500 ml flask equipped with a mechanical stirring condenser and thermometer and charged with ethyl acetate (500 ml) and dichloromethane (1500 ml) was added crude olmesartan medoxomil (50 g). The suspension was slowly heated to a temperature ranging from about 50° C. to about 55° C. and maintained for about 1 to 2 hours to obtain a clear solution. The solution was then stirred at the same temperature for 1 hour and any insolubles were removed by filtration. The clear filtrate was slowly cooled to room temperature and then further cooled to 10° C. to 20° C. The precipitate was filtered on a Buckner funnel and washed with ethyl acetate (50 ml) was a solid enriched in olmesartan medoxomil. The filtered product was dried to provide pure olmesartan medoxomil (36 g) having a purity of 99.82% and a content of free olmesartan of 0.08% as determined by HPLC.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A process for purifying olmesartan medoxomil comprising (a) dissolving olmesartan medoxomil in a solvent system comprising a ketone and at least one solvent selected from the group consisting of an alcohol-containing solvent, an ester-containing solvent and mixtures thereof to obtain a solution; and (b) recovering substantially pure olmesartan medoxomil.

2. The process of claim 1, wherein the solution is formed at an elevated temperature.

3. The process of claim 1, wherein step (a) comprises heating the olmesartan medoxomil dissolved in the solvent system to a temperature of about 30° C. to about 100° C.

4. The process of claim 1, wherein the solvent system comprises a ketone and an ester-containing solvent.

5. The process of claim 4, wherein the ketone is a ketone having 3 to about 12 carbon atoms and the ester-containing solvent is an acetate.

6. The process of claim 1, wherein the solvent system comprises acetone and ethyl acetate.

7. The process of claim 1, wherein the concentration of the ketone in the solution is about 5 to about 10% weight per volume (w/v) and the concentration of the ester-containing solvent in the solution is about 10 to about 20% w/v.

8. The process of claim 2, wherein the step (b) comprises precipitating the olmesartan medoxomil; and (d) recovering substantially pure olmesartan medoxomil

9. The process of claim 8, wherein precipitating is induced by cooling the solution from the elevated temperature.

10. The process of claim 9, wherein the step of precipitating comprises cooling the solution to a temperature of about 0° C. to about 30° C.

11. The process of claim 9, wherein the step of precipitating comprises cooling the solution to a temperature of about 10° C. to about 25° C.

12. The process of claim 8, further comprising drying the purified olmesartan medoxomil.

13. The process of claim 1, further comprising heating the solution to an elevated temperature; cooling the solution to induce precipitation of the olmesartan medoxomil; and recovering substantially pure olmesartan medoxomil.

14. The process of claim 1, wherein the substantially pure olmesartan medoxomil is obtained in a purity of greater than or equal to about 98%.

15. The process of claim 14, wherein the substantially pure olmesartan medoxomil is obtained in a purity of greater than or equal to about 99%.

16. The process of claim 15, wherein the substantially pure olmesartan medoxomil is obtained in a purity of greater than or equal to about 99.5%.

17. The process of claim 16, wherein the substantially pure olmesartan medoxomil contains less than about 0.5% of free olmesartan.

18. The process of claim 17, wherein the substantially pure olmesartan medoxomil contains less than about 0.1% of free olmesartan.

19. Olmesartan medoxomil produced by the process of claim 1.

20. The olmesartan medoxomil of claim 19, having a purity of greater than or equal to about 99%.

21. Olmesartan medoxomil having a purity of greater than or equal to about 98%.

22. The olmesartan medoxomil of claim 21, having a purity of greater than or equal to about 99%.

23. The olmesartan medoxomil of claim 21, having a purity of greater than or equal to about 99.5%.

24. The olmesartan medoxomil of claim 21, having less than about 0.5% of free olmesartan.

25. The olmesartan medoxomil of claim 21, having less than about 0.1% of free olmesartan.

26. A pharmaceutical composition comprising a therapeutically effective amount of the olmesartan medoxomil of claim 21, and one or more pharmaceutically acceptable excipients.

27. The pharmaceutical composition of claim 26, wherein the olmesartan medoxomil is a micronized olmesartan medoxomil having a D₅₀ and D₉₀ particle size of less than about about 200 microns.

28. The pharmaceutical composition of claim 26, wherein the olmesartan medoxomil is a micronized olmesartan medoxomil having a D₅₀ and D₉₀ particle size of less than about 150 microns.

29. The pharmaceutical composition of claim 26, wherein the olmesartan medoxomil is a micronized olmesartan medoxomil having a D₅₀ and D₉₀ particle size of less than about 50 microns.

30. The pharmaceutical composition of claim 26, wherein the olmesartan medoxomil is a micronized olmesartan medoxomil having a D₅₀ and D₉₀ particle size of less than about 15 microns.

31. A process for purifying olmesartan medoxomil, the process comprising (a) providing a solution comprising olmesartan medoxomil in a solvent system comprising a halogenated hydrocarbon-containing solvent and an ester-containing solvent; and (b) recovering substantially pure olmesartan medoxomil.

32. The process of claim 31, wherein the halogenated hydrocarbon-containing solvent is dichloromethane and the ester-containing solvent is ethyl acetate.

33. The process of claim 32, further comprising heating the solution to an elevated temperature; cooling the solution to induce precipitation of the olmesartan medoxomil; and recovering substantially pure olmesartan medoxomil.
